(19) United States
(12) Patent Application Publication
(10) Pub. No.: US 2023/0240690 A1
(43) Pub. Date: Aug. 3, 2023

(54) Device For Constricting Vessels

(71) Applicant: DaisyGrip GmbH, Rostock (DE)

(72) Inventors: Konstantin Altrichter, Kavelstorf (DE); Karl Hartmann, Elmenhorst-Lichtenhagendorf (DE)

(73) Assignee: DaisyGrip GmbH, Rostock (DE)

(21) Appl. No.: 18/192,809

(22) Filed: Mar. 30, 2023

Related U.S. Application Data
(63) Continuation of application No. 16/649,219, filed on Mar. 20, 2020.

(30) Foreign Application Priority Data
Sep. 20, 2017 (DE) 10 2017 121 812.2

(51) Int. Cl. A61B 17/132; A61B 5/022

(52) U.S. Cl. CPC A61B 17/1327; A61B 5/02233; A61B 2090/081

(57) ABSTRACT

A device constricts vessels and includes a constricting belt and a lock. One end of the constricting belt is arranged in a fixed manner in the lock and one end of the constricting belt is movably arranged in the lock. The constricting belt and the lock include a material having a smooth surface. In a closed state, the lock is a smooth, cube-shaped or elongated body, which includes no further external openings or projections apart from the feedings for the constricting belt.

### CROSS-REFERENCE TO RELATED APPLICATIONS

This application is a continuation of U.S. patent application Ser. No. 16/649,219, filed Mar. 20, 2020, which is the National Stage of International Patent Application No. PCT/EP2018/075368, filed Sep. 19, 2018, which claims priority to and all the advantages of German Patent Application No. DE 10 2017 121 812.2, filed Sep. 20, 2017, the entire contents of each of which are hereby incorporated by reference.

### TECHNICAL FIELD

This invention relates to a hygienic device for constricting vessels (tourniquet) with a constricting belt, which is connected by means of a lock to a closed circular belt. The tourniquet according to the invention, also termed constriction tube or vein constrictor, is used in medicine.

### BACKGROUND

Tourniquets are used to achieve a compression to create a constriction pressure in blood vessels, for example, e.g. when collecting blood or establishing other accesses, or to enable temporary constriction of a blood vessel for blood pressure measurement.

Without proper disinfection, the daily use of tourniquets in routine blood collection inevitably leads to germ transmission. In Germany alone, nosocomial infections (infections contracted in hospital) have fatal consequences for 40,000 patients every year.

Prior to each blood collection, a tourniquet is applied. Tensioning of the tourniquet leads to a build-up of blood in the veins, due to which these become more visible and can be punctured more easily with a cannula. Such a procedure often takes place several times a day in the case of hospital patients. Cleaning is necessary after every application.

All solutions to date for tourniquets offer a large surface for germs to accumulate. Circular cuffs, for example, are placed around a patient's arm and the ends tied together. Some solutions also require precise insertion of a belt through a tongue. Other solutions use a mechanical lock, which likewise requires active joining and offers a number of accumulation surfaces for germs due to the plug-in connection. In particular, the nature of previous belts contributes to the microbial load.

The previous options for returning these tourniquets to a low-germ state—for cleaning them—necessitate up to two hours in a sterilisation unit or autoclave. Other options, such as liquid disinfectants, do not achieve an adequate cleaning state due to the design of the tourniquets, their accumulation surfaces and/or the belt material.

The most widespread tourniquets, such as described in U.S. Pat. No. 5,535,485A, consist chiefly of an elastic, terry-like belt and a two-part lock element. Due to the mechanical configuration of the lock, the surface has very many indentations and movable parts, which constitute germ reservoirs. The previous cleaning instruction of the manufacturers of such tourniquets for ensuring hospital hygiene provides that these tourniquets are treated in an autoclave unit. This requires a roughly 2-hour procedure of pressure, steam, heat and radiation treatment. In particular, the constricting belt and the lock parts represent germ reservoirs, as both have a large, rough and thus hard to clean surface. Tourniquets of this kind must be treated accordingly in the autoclave unit to facilitate a release from germs. Macroscopic, including visually visible impurities are difficult to impossible to remove from the belt, textile portions and a woven structure rule out other cleaning options such as wiping bloodstains away.

The patent DE 19650969 C1 thus discloses a tourniquet with a strap and a clasp, which has two coupling elements. A first coupling element has a hook-shaped latching element, which engages detachably in a second coupling element. The coupling elements have normal clamping surfaces for clamping the strap.

In patent EP 633747 B1, a constricting device for parts of the body with a constricting belt and a lock housing is disclosed, in which one end of the constricting belt is connected to the lock housing and the free end is guided through the lock housing. A rocker serves to clamp the belt fixedly in the lock housing. Slow deconstriction is facilitated through pressure on an actuating part.

A tourniquet strapping system such as shown in WO 2016/172599 A1 describes a buckle for a constriction band by means of which the blood flow is to be suppressed by pressure on a blood vessel in order to avoid extreme blood loss. To maintain this state, magnetic retaining devices are used among other things. By rotation of a bar, the constriction band connected to this is tied tightly around the body part to be bound. The bar is then fixed on the buckle by a tongue, knobs or a magnetic securing. Tensioning accordingly takes place independently of the magnetic securing. The securing can only maintain the toggle pressure built up previously by a bar and prevent back-rotation of the bar. The design of the belt and of the tensioning and retaining mechanism with many individual parts and concealed edges constitutes germ reservoirs that oppose low-germ working in daily clinical practice.

An expanded tourniquet strapping system as described in US 2014/0277103 A1 is restricted, furthermore, to use in an emergency. Even an additional (magnetic) lock of the strap, into which the tensioning mechanism is again not integrated, does not lead to improved cleanability/hygiene. A belt only with the described lock cannot be tensioned/slackened for blood congestion. As already specifically described in the patent, it would not be for use as a tourniquet, but a simple strap for holding objects together. This circumstance is due to the basic idea of the invention, which concentrates on the military emergency treatment.

In CN 205831862 U, an elastic belt is provided at both ends of the circular belt with a lock part respectively. The two lock parts are equipped with magnets. The user must actively join and hook up the lock parts and then bring the magnets into a position in which the magnetic forces can act as a securing. Here also the magnet acts only for securing in the constriction position and supports holding in the tensioned state. The hook-shaped undercuts necessary for holding in position create opportunities for germs to accumulate, due to which the belt with the lock parts can scarcely be processed in a hygienic manner for on-site disinfection.

In the patent publication CN 104207817 A, a constriction device is described that can also be of silicone, the holes of which through the lock necessitated by the belt mechanism also argue against simple cleaning, however, like the latching of the lock, due to which new openings and germ reservoirs arise on the lever/pin. Furthermore, application to the patient has to be carried out using two hands.

### SUMMARY

The aim of the invention is to design the lock and the constricting belt of a tourniquet in such a way that these can be used as hygienic reusable vein constrictors in that overall a simpler disinfection possibility, such as a spray/wipe disinfection on site, can be carried out to prevent germ transmission. Moreover, the aim of the invention is that the lock elements autonomously find their locking position even in the event of inaccurate joining. The joining and the release of the lock should be able to be carried out with one hand.

This aim is achieved by the features disclosed in the claims.

The device according to the invention for constricting vessels comprises a constricting belt with a width of between 0.5 and 50 cm, preferably between 1 and 10 cm for one embodiment, for example as a tourniquet, and between 7 and 30 cm for another embodiment, for example as a blood pressure cuff, and a lock which contains two lock elements, wherein one end of the constricting belt is fixedly connected to one lock element and the other lock element is arranged movably on the constricting belt. The constricting belt and the lock consist of a material with a smooth surface. In the closed state, the lock is a smooth, cube-shaped or elongated body, which has no further external openings and, due to flush edges between the lock elements, has no external projections apart from the feedings for the constricting belt. Flush should also be understood as meaning that the finishing edges are largely flush, i.e. they can also run conically or convexly, for example. The constricting belt consists of a textile-free material and the constricting belt and/or the lock are provided with an anti-friction coating. The coating of the lock and/or of the constricting belt consists in one embodiment in particular of a fluorination of the surface. Any equivalent surface sealing that would achieve an equivalent improvement of the sliding properties can also take place, however.

Textiles attain an elongation maximum due to the loop arrangement. Constriction effects thereby occur more rapidly, which cause soft tissue bruising and thromboses. Due to the intermolecular effects, a rubber belt has no such pronounced maximum, constriction is prevented thereby and softer adjustment options for the constriction pressure can be realised.

Apart from plastic, aluminium, stainless steel or the like can be materials for the lock. Instead of silicone, the constricting belt can also consist of another elastic material with a closed surface, such as e.g. latex, thermoplastic elastomers, Teflon or similar.

In one embodiment there is provided in one lock element a locking device for the constricting belt. The movable lock element is magnetically connectable to the fixed lock element.

For another preferred embodiment, one lock element is provided on one side facing the other lock element on at least one, preferably at least two external edges with chamfers and grooves and the other lock element is provided on at least one, preferably at least two external edges with webs and tongues and both lock elements are executed flush with one another on the external edges in the closed state. The chamfers of one lock element correspond with the webs of the other lock element and the grooves of one lock element correspond with the tongues of the other lock element.

For another embodiment a sensor system for determining vital parameters, such as heart rate/pulse, blood pressure, blood oxygen content, and/or an activity cleaning state, for example, is integrated in the constricting belt, such as, for example, status by magnetic sensor: open/closed, proximity to cleaning location, strain gauge, and/or in the lock, here a magnetic sensor, light/acoustic/elongation sensor can be used, for example.

Another embodiment integrates a transmission and receiving unit in the constricting belt and/or in the lock, which unit facilitates forwarding of information determined by the sensor system, for example.

The lock according to the invention for a device for constricting vessels has at least two lock elements, which are connected detachably to one another. One lock element is arranged fixedly with a constricting belt and the other lock element is arranged movably on the constricting belt. The lock elements are provided according to the claim with chamfers and grooves and corresponding webs and tongues and are executed flush with one another on their external edges. The webs with the tongues or the chamfers with the grooves are connected to the movable or the fixed lock element. The webs are arranged on the lock element at an angle to the lock element. It is conceivable that both webs are inclined at an angle other than 90 degrees in an identical direction or in an opposite direction. It is then necessary that the chamfers are designed accordingly, so that webs and chamfers and grooves and tongues can engage in one another. The angle between the webs and the lock elements should expediently be between 30 and 150 degrees, preferably approx. 80 degrees. The detachable connection can take place by magnetic locking, for example.

In one embodiment the webs are executed in an arcuate shape (curved) in the direction of the opposing lock element and the chamfers are designed corresponding to the webs.

For one preferred embodiment the webs on the lock element are somewhat wider in the manner that the webs have a base and a tip, wherein the base is at least 0.1 mm, preferably 0.5 mm to 5 mm wider than the tip, and the chamfers are designed corresponding to the webs, so that an easily sliding interplay of both lock elements with one another is guaranteed, which can be improved further by at least one flexible web.

Connected to the lock elements are magnets, which act with their magnetic fields between the two lock elements and which pull the lock elements into one another by their opposite polarity, due to which locking takes place. The magnetic force of the connected lock elements can additionally initiate the locking mechanism and block the belt (in one direction) when the lock is closed. A repellent magnetic field can act on the fourth external edge of the first lock element, whereby a release of the two lock elements from one another is accelerated. Furthermore, the magnetic field can also be operated electronically, in order to adjust the field strength of the magnetic field targetedly, for example, or to be able to adjust the sensor system. Switching off of the electronics, for example following use, can also be enabled thus.

For one particular embodiment, a locking device with a surface relief is arranged inside one lock element, which device is a custom-fit counterpart to a constricting belt provided with a corresponding surface relief.

A lever or a wheel can additionally be arranged on the lock in order to build up additional tensioning of the constricting belt.

A blood pressure cuff can likewise use a device for constricting vessels according to the inventive solution and be connected by an inventive lock.

The device according to the invention for constricting vessels permits a simpler use sequence with improved hygiene properties. The improved usability for the user is facilitated by single-handed operation in the entire process. Due to the magnetic force in the lock, the overall locking mechanism is executed quasi autonomously. The lock finds itself, so to speak. Here the magnets can be incorporated in the lock, in order not to form the surface of the lock.

The opening of the lock commences by a movement directed transversely to the constricting belt, for example a simple swiping movement of the thumb (as when unlocking a smartphone). Due to the web shape the opening process can likewise be formed in a curved movement. After overcoming the magnetic potential maximum, which serves to secure the lock, repellent forces can support the opening process of the lock.

Due to the smooth parts of the constricting belt and the lock, faster cleaning and the release of germs located on the tourniquet are considerably simplified and improved. The creation of a germ reservoir is prevented by the transfer of the moving external lock parts into the interior, so that there are no prominent/uneven places for germs to accumulate. A device in the interior region can additionally ensure that the tourniquet cleans itself even without external measures. One embodiment would be a deflection roller of ionic-bactericidal materials. Autoclaving or other technical sterilisation measures are no longer necessary.

It is also possible by the integration of suitable electronics to detect vital functions of the patient as well as the cleaning status of the tourniquet and to transmit them, for example by radio, to a central system.

### BRIEF DESCRIPTION OF THE DRAWINGS

The invention is explained in greater detail by means of exemplary embodiments. With regard to these,

FIG. 1 shows a device according to the invention for constricting vessels in a view in perspective,

FIG. 2 shows the device for constricting vessels in another view in perspective,

FIG. 3 shows a side view of FIG. 1,

FIG. 4 shows a cross section of a closed lock,

FIG. 5 shows a blood pressure cuff with a device for constricting vessels,

FIG. 6 shows the lock in a view in perspective, and

FIG. 7 shows a cross section of the lock.

### DETAILED DESCRIPTION

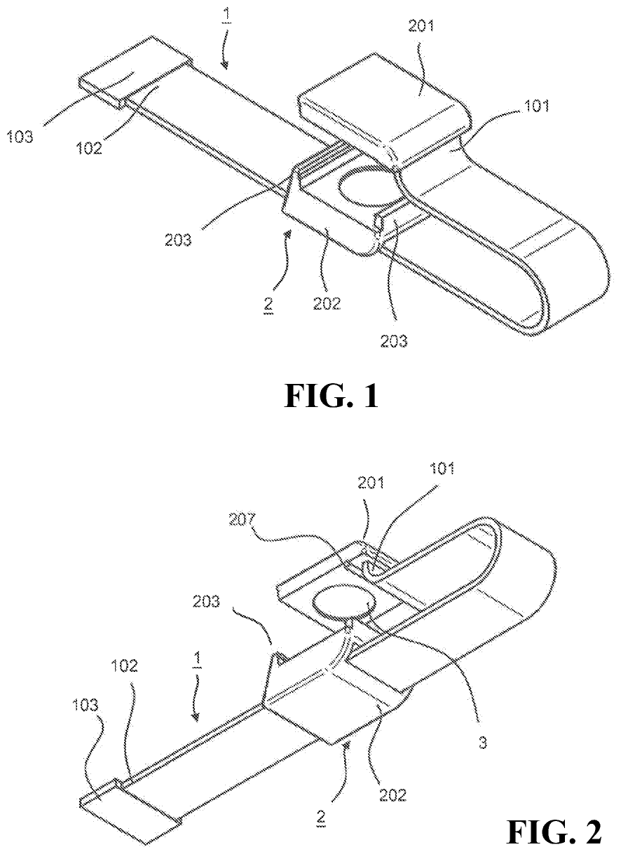

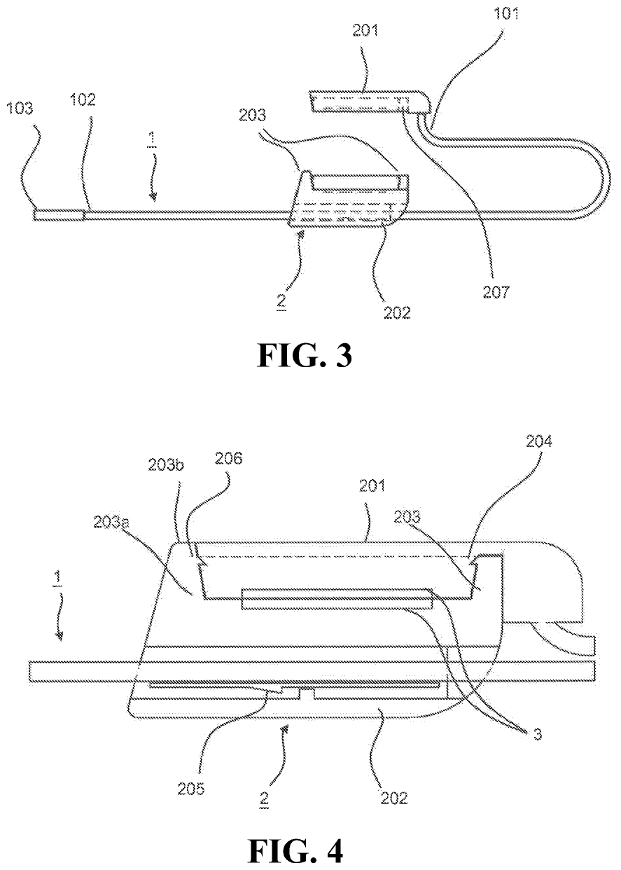

FIGS. 1 to 3 show the device according to the invention for constricting vessels in different views in perspective and in a side view with an open lock.

The device according to the invention for constricting vessels consists of an elastic constricting belt 1 with a smooth surface, for example of a textile-free silicone material. The constricting belt 1 is connected to a lock 2, so that it can be connected to a closed circular belt. The lock 2 consists of at least two lock elements 201 and 202, which are connected detachably by means of a magnetic lock. Furthermore, apart from the concluding edge between the lock elements, the lock 2 in the closed state is a smooth, cube-shaped or elongated body with preferably rounded edges, which has no other external openings or projections apart from the feedings for the constricting belt 1, as the edges between a first, fixed lock element 201 and a second, movable lock element 202 end flush. Other concluding edges, e.g. between production-related upper and lower shell, are negligible as openings.

At a first end 101 of the constricting belt, the first, upper lock element 201 is arranged in the manner that the constricting belt 1 is threaded into this lock element 201 and secured by gluing, pressure, screws or magnetically, for example, so that the constricting belt 1 is connected fixedly to the lock element 201. The second end 102 of the constricting belt 1 is led through the second, lower, movable lock element 202 in a through-opening provided for this and delimited at its end 102 by a stop mechanism 103, so that the lock element 202, which is movable on the smooth elastic constricting belt 1, cannot slide off the constricting belt 1. In the second lock element 202 there is provided for the constricting belt 1 a locking device 205, for example in the form of a slide rail, with a locking mechanism, for example a rocker, a wedge, a tongue, a roughened surface or a pressure roll. The movable lock element 202 is magnetically connectable to the fixed lock element 201 and locks mechanically automatically. Following the connection of the two lock elements 201, 202 to one another, the locking device is actuated automatically by the process of tensioning around an object.

A magnetic locking mechanism is used for the magnetic connection of the two lock elements 201, 202. Due to the particular design according to the invention of the lock 2, one-handed closing, tensioning and opening of the constricting belt 1 is thus made possible. In

FIG. 4 a closed lock 2 is shown. A magnet 3 is incorporated centrally and directed towards one another in each lock part 201, 202.

One of the two lock elements 201, 202, for example the second, lower lock element 202, is provided on several, e.g. two external edges on the side facing the other lock element 201 with webs 203 transversely to the constricting belt 1. The webs 203 are provided at their upper end pointing into the interior of the lock with tongues 206. The first lock element 201 is accordingly designed so that the two lock elements 201, 202 can engage in one another. To this end the first lock element 201 is provided on two external edges, for example, with a front and a rear chamfer 207. On two surfaces of the chamfers 207 pointing outwards, grooves 204 are incorporated, which correspond with the tongues 206. The tongues 206 engage in the grooves 204 and enable additional securing in this way. The third and fourth external edge, preferably in a longitudinal direction to the constricting belt 1, is configured flush with the other lock element 201, 202 respectively. The webs 203 and the tongues 206 can be connected to the movable or the fixed lock element 201, 202. The same applies to the grooves 204 and the chamfers 207 in a corresponding manner to the tongues 206 and webs 203. The webs 203 are arranged on the lock element 201, 202 in this exemplary embodiment at a right angle to the lock element 201, 202. The angle between the webs 203 and the lock elements 201, 202 should expediently be between 30 and 150 degrees, preferably approx. 80 degrees. For better handling of the lock 2, they can be somewhat wider towards the lock element 201, 202 in the manner that the webs 203 on the lock element 201, 202 have a base 203a and a tip 203b, wherein the base 203a is at least 0.1 mm, preferably 0.5 mm to 5 mm wider than the tip 203b. The chamfers 207 are designed correspondingly, so that in any event an easily sliding interplay of the two lock elements 201, 202 with one another is guaranteed. Due to the magnets 3 located in the interior of the lock 2, the two lock elements 201, 202 are self-locating when they come into the sphere of influence of the magnetic field. One-handed operation (application, tensioning, slackening and release) of the constricting belt 1 is facilitated. To release the two lock elements 201, 202 from one another the upper lock element 201 is pushed, for example using the thumb, in the direction in which no web 203 is present on the lower lock element 202, whereby several directions are conceivable. The movement is carried out until the attracting influence of the magnets 3 decreases and the two lock elements 201, 202 separate from one another due to the (possible) repellent effect developing.

The constricting belt 1 and/or the lock 2 are provided with a smoothing coating. The extraordinary smoothness of the constricting belt and/or of the lock is achieved by the special coating. In particular, fluorination of the surface is possible for this, but any other anti-friction coating, for example using silicon oxides or similar, is also possible. In the fluorination, hydrogen atoms are detached from the side chains and replaced by fluorine, whereby the surface properties change. On the one hand the adhesive and dynamic friction and the adhesiveness are reduced significantly, while on the other wettability is increased, due to which polar liquids, such as in liquid disinfection, form a special surface film and no longer tend to form local drops due to the characteristic polarity. Thus better sliding properties develop with flawless liquid films, which leads in the case of the tourniquet to better application to the skin and improved cleanability due to wetting of the overall surface and thus the release of germs at every point. Due to this, germs are prevented from remaining on the unit following the use of the tourniquet and posing a risk of infection for the next patient. This coating can be checked by a tilt test, in which the coated material and the uncoated material lie on a plane that is located at 0° and can be inclined fluidly/continuously up to 100°. The constricting belt described here reaches less than 35° for hygiene purposes when it begins to slide.

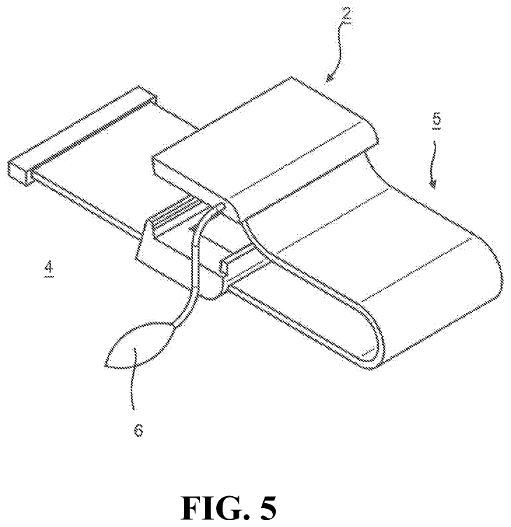

FIG. 5 shows a blood pressure cuff 4 with a device for constricting vessels as another exemplary embodiment. To this end a smooth belt 5 can be connected in the manner described using the lock 2 described in detail above, which can be dimensioned for this application larger than for the constricting belt 1. The variable pressure setting customary according to the prior art can be achieved by different mechanisms, only the pneumatic pressure rise by means of a pump is named here as the most common.

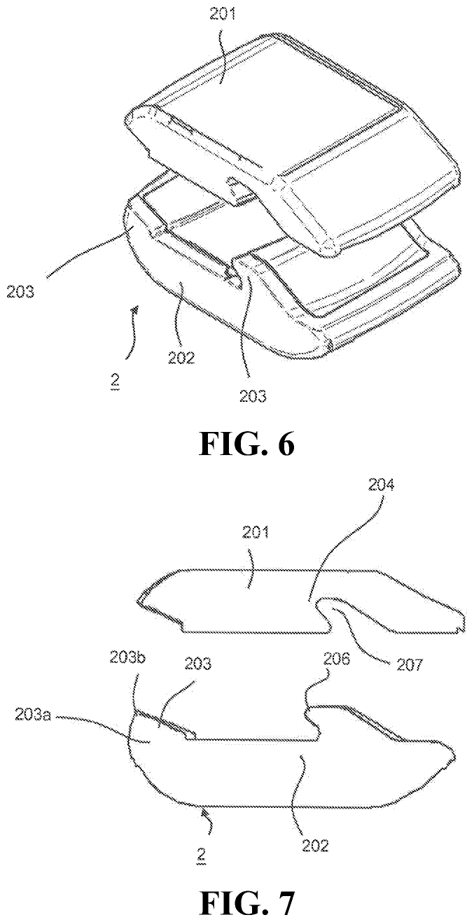

FIGS. 6 and 7 show the lock 2 with arcuate webs 203, which engage in correspondingly shaped grooves 204, whereby the lock elements 201, 202 are held better in one another due to the force transmission in the event of tension. The magnets 3 in this embodiment are located inside the lock 2. The rounding of the arcuate webs 203 engaging in one another contributes to the shear forces acting in a larger segment of a circle than in the case of exclusively straight edge guidance. A more secure holding of the lock elements 201, 202 to one another is guaranteed hereby and the retaining forces of the magnetic potential maximum are supported.

It is conceivable furthermore to form the tourniquet without magnets to facilitate use in the vicinity of a magnetic resonance scanner. For this, locks 2 can also be used that are only conceived for tightening and releasing, and possibly not even for opening, but are only placed over the arm, but which have the hygienic properties of said invention and have a corresponding anti-friction coating.

It is also conceivable to integrate sensors into the tourniquet described above, such as e.g. strain gauges or fibres that resistively perceive length changes in the constricting belt 1 or in the belt 5. The measurement of blood pressure or other vital parameters can be carried out by means of the existing tensioning mechanism in that the necessary pretensioning is generated by the inventive device for constricting vessels. The pretensioning then no longer needs to be varied by pumps. The blood pressure among other things can be detected by a sensor system for determining vital parameters that is integrated into the constricting belt 1 and/or in the lock 2, which constitutes a new type of measuring method for blood pressure. A suitable sensor system in the tourniquet or the blood pressure cuff can likewise help to detect the cleaning status.

It is also conceivable that a transmission and receiving unit is integrated in the constricting belt 1 and/or in the lock 2, which unit enables information to be forwarded. By communication with a corresponding base station the hygienic state of the tourniquet and the vital parameters can be transmitted and documented. This in turn contributes to the adherence to hygiene guidelines. For this a display unit can be integrated in the constricting belt 1 or the lock 2.

Due to modified properties of the constricting belt 1, for example in the form of a (toothed) surface relief and reduced extensibility, a higher constriction pressure can be built up in order to stop the complete blood flow in arteries and veins especially in emergency situations. To this end the slide rail of the locking device 205 inside the second lock element 202 must be arranged with a custom-fit counterpart corresponding to the surface relief. Additional tensioning of the constricting belt 1 can take place following locking via a lever or a wheel, for example, on the lock 2. Hygiene plays a subordinate role in this extreme situation. What is important is that one-handed operation due to the self-locating magnetic lock represents an enormous survival advantage.

The device for constricting vessels and/or the lock 2 for the device for constricting vessels and/or the blood pressure cuff 4 is cleaned by the following steps:
Application of a cleaning agent or disinfectant by means of a disinfectant dispenser. The disinfectant dispenser can be mounted fixedly on a wall for this or a mobile bottle of disinfectant can be used.
Immersion in a cleaning or disinfectant bath can be undertaken alternatively
or treatment with a cloth soaked in a cleaning agent or disinfectant can take place,
the disinfecting liquid should act for at least 1 s to a maximum of 10 mins, preferably 30 s.
Finally disinfecting liquid residues that are still present with one of the three application methods are removed by wiping, for example with a disposable cloth.

A cleaning device that is stationary or portable, for example arranged on a tray, and which is not to be depicted further here, can be used for the method. The tourniquet is transported by this device in that rollers or rolls, for example, provide the propulsion. The rollers or rolls are provided with sponges saturated in disinfectant. Liquid residues are removed by a second pair of sponges, rollers or rolls. The transport can be continuous or discontinuous. Another option consists in the constricting belt 1 and the lock 2 or also the blood pressure cuff 4 being placed in a recess provided for this, which consists of soft sponges, for example. Either the rollers or rolls are moved across the tourniquet here also or the tourniquet is pulled through the device.

Since the devices for constricting vessels described in detail above are exemplary embodiments, they can be modified in a usual manner by the expert in a wide scope without departing from the field of the invention. In particular, the specific configurations of the lock 2 and the lock elements 201, 202 can follow in a form other than that described here. The constricting belt 1, in particular the blood pressure cuff 4, can likewise be configured in a different form if this is necessary for reasons of space, design or technological reasons. Furthermore, the use of the indefinite article “a” does not exclude the possibility of the multiple presence of the relevant features.

### REFERENCE SIGNS

1 Constricting belt
101 First end of the constricting belt 1
102 Second end of the constricting belt 1
103 Stop mechanism

### 2 Lock

201 First lock element
202 Second lock element

### 203 Web

### 203a Base of web

### 203b Tip of web

### 204 Groove

205 Locking device

### 206 Tongue

### 207 Chamfers

### 3 Magnet

4 Blood pressure cuff

### 5 Belt

### 6 Pump

What is claimed is:

1. A constricting belt for a hygienic device for constricting vessels, wherein the constricting belt comprises a textile-free material and is provided with an anti-friction coating.

2. The constricting belt according to claim 1, wherein the anti-friction coating seals a surface of the constricting belt. The constricting belt according to claim 1, wherein the anti-friction coating is provided such that in a tilt test the constricting belt begins to slide at less than 35°.

4. The constricting belt according to claim 1, wherein the anti-friction coating of the constricting belt results from fluorination of a surface of the constriction belt.

5. The constricting belt according to claim 1, wherein the constricting belt is made of an elastic material with a closed surface.

6. The constricting belt according to claim 5, wherein the constricting belt is made of silicone or latex.

7. The constricting belt according to claim 1, wherein the constricting belt is provided with a surface relief.

7. The constricting belt according to claim 7, wherein a lock element is arranged movably on the constricting belt, and a locking device is arranged within the lock element, wherein the locking device comprises a surface relief corresponding to the surface relief of the constricting belt.

9. The constricting belt according to claim 1, wherein the constricting belt has a first end and a second end, and is adapted to be connected to a closed circular belt.

10. The constricting belt according to claim 1, wherein the constricting belt has a first end, and wherein the first end is fixedly connected to a first lock element.

11. The constricting belt according to claim 10, wherein the first end of the constricting belt is threaded into the first lock element and secured thereto.

12. The constricting belt according to claim 1, wherein a second lock element is arranged movably on the constricting belt.

13. The constricting belt according to claim 1, wherein a sensor system for determining at least one of vital parameters and a cleaning condition is integrated into the constricting belt.

14. The constricting belt according to claim 1, wherein at least one of a transmitting unit and a receiving unit is integrated into the constricting belt, which unit enables information to be forwarded.

15. The constricting belt according to claim 1, wherein the constricting belt comprises a material with a smooth surface.

16. The constricting belt according to claim 1, wherein one end of the constricting belt is delimited by a stop mechanism.

17. The constricting belt according to claim 1, comprising a lock for connecting the constricting belt to form a closed circular belt, wherein the lock comprises a lock element which comprises a material with a smooth surface.

18. The constricting belt according to claim 17, wherein the lock comprises an anti-friction coating resulting from fluorination of a surface of the lock.

19. A method for cleaning a constricting belt for a hygienic device for constricting vessels, wherein the constricting belt comprises a textile-free material and is provided with an anti-friction coating, and wherein a roller or a roll, which has received disinfectant, is moved over the constricting belt.

20. A method for operating a constricting belt of a vascular tourniquet, comprising:
providing a constricting belt with a first end and a second end, wherein the constricting, belt comprises a textile-free material and is provided with an anti-friction coating;
connecting the constricting belt to a closed circular belt; and
creating a constriction pressure with the constricting belt.
